(19) United States
(12) Patent Application Publication
(10) Pub. No.: US 2024/0122848 A1
(43) Pub. Date: Apr. 18, 2024

(54) APPETITE SUPPRESSANT

(71) Applicant: Milton S. JACKSON, Campbellsville, KY (US)

(72) Inventor: Milton S. JACKSON, Campbellsville, KY (US)

(21) Appl. No.: 18/392,777

(22) Filed: Dec. 21, 2023

Related U.S. Application Data
(62) Division of application No. 16/614,977, filed on Nov. 19, 2019.
(60) Provisional application No. 62/508,929, filed on May 19, 2017.

(51) Int. Cl. A61K 9/00; A61K 31/167; A61K 31/55; A61K 47/10; A61K 47/38

(52) U.S. Cl. CPC A61K 9/0073; A61K 9/0043; A61K 31/167; A61K 31/55; A61K 47/10; A61K 47/38

(57) ABSTRACT

Disclosed is a pharmaceutical composition useful as an appetite suppressant. Disclosed also is a process for the suppression of appetite and/or for the treatment and/or prevention of obesity.

### RELATED APPLICATIONS

This application is a divisional of U.S. patent application Ser. No. 16/614,977, filed Nov. 19, 2019, which is a 35 U.S.C. § 371 application of International Application Serial No. PCT/US/2018/032978, filed on May 16, 2018, which claims the benefit of priority of U.S. Provisional Application No. 62/508,929, filed May 19, 2017. The entire content of the applications referenced above are hereby incorporated by reference herein.

### FIELD

The present disclosure relates to a pharmaceutical composition useful as an appetite suppressant. The present disclosure also relates to a process for the suppression of appetite and/or for the treatment and/or prevention of obesity.

### BACKGROUND

Obesity is the greatest health problem in the United States and one of the greatest health problems worldwide. There are one hundred million morbidly obese individuals in the United States. There are also one hundred million overweight individuals in the United States. The yearly cost from obesity is estimated to be on the order of $200,000,000,000 to $400,000,000,000. Obesity and related illnesses have tremendous physical, psychological, and financial impact. Present treatments for obesity are expensive, inadequately effective, and have significant risks and side-effects. Obesity is a growing epidemic despite all of the advances of modern medicine.

Obesity is a consequence of a generally sedentary lifestyle and the fact that individuals “enjoy” an abundance of unhealthy foods. Approximately one-third of the population of the United States is overweight, and another one-third of the population is either obese or morbidly obese. Obesity is the second-leading cause of preventable death after tobacco abuse. In addition to the large number of medical problems caused by obesity, being overweight has a very significant negative impact on quality of life. Hundreds of billions of dollars are spent treating obesity, but the epidemic continues to increase. Dietary and activity changes with both medical and surgical care are not controlling this situation. Modern medicine has failed to control this massive problem.

Obese patients are unable to maintain a healthy lifestyle with necessary increases in activity and exercise along with dietary changes to lose weight. Current and available medical treatments are marginally effective and have significant costs and side effects/risks associated with them. Surgeries for obesity are generally more effective than medical treatments, but have enormous costs with both short- and long-term risks and complications. Despite the fact that obesity is a growing epidemic, medical professionals are spending less time treating and following these patients due to the inadequacy of treatment options and the time requirements to do so. It is unfortunately ironic that the treatments for the comorbidities of obesity are more effective than treating the weight gain and eating problems.

It is a simple but unfortunate fact that weight gain and obesity occur when a person consumes more calories than they burn. The accumulation of fat is a consequence of environmental (eating, activity, behavioral, and social) and genetic factors. Being overweight is defined as having a BMI>30 (BMI=wt. in kg./ht. in m. squared), and morbidly obese is BMI>35. Ideal weight according to the Metropolitan Life Tables (standard source) is BMI<25. Worldwide, 2,500,000 deaths are attributed to obesity yearly.

Mortality rate for a patient with BMI>40 is double that of a normal weight individual. Surgical treatments are indicated for patients failing medical treatment, for BMI>35 with comorbidities, or for BMI>40. It is extremely difficult to quantify or objectify the impact that obesity has on quality of life and lifestyle. Deaths from obesity are due to the comorbid conditions and complications of the excess weight. Obesity causes or contributes to diabetes and insulin resistance, hypertension, heart disease, vascular disease, strokes, dyslipidemia, liver disease, gastroesophageal reflux, urinary incontinence, cellulitis and other infections, obstructive pulmonary disease and respiratory insufficiency, sleep apnea, sexual and other hormone dysfunction, depression, gallbladder disease and dysfunction, and a multitude of orthopedic problems including neck, back, hip, knee, and foot issues and pain. Obesity is a significant contributing factor in cancers of the uterus, breast, ovaries, prostate, colon, esophagus, pancreas, and thyroid, and possibly others. Other medical conditions related to obesity include Cushing's disease, polycystic ovary syndrome, “metabolic syndrome”, and psychogenic or genetic eating disorders.

Obesity is obviously a horrible problem, not only at a personal level, but as a society. It takes a long time to gain significant weight, and unfortunately it takes very significant lifestyle changes over a long time to lose this weight. Even a 10-15% weight loss has health benefits. Long-term and actually life-long changes and commitments to improve eating and activity are required to lose weight and regain good health and well-being. 3500 kcals is the equivalent of one pound of body weight. For weight loss, the typical diet includes 1200-1800 kcals/day. This should include quality nutrition sources of proteins, vitamins, complex carbohydrates, fiber, and fluids. Learning to eat the correct amounts of the right foods is imperative to losing weight and regaining health. Burning calories through increased activity and exercise also results in burned calories through an increase in metabolism. Overall weight loss depends on more calories burned than consumed. This must be maintained for some time to have the success of weight loss and maintenance and regain the health benefits of the lower weight. Looking better, feeling better, and living better and longer are the results.

Existing medical and surgical treatments for obesity are not “fixes” or “answers” to the problem. They are but tools to help achieve the goal of weight loss and regaining health benefits. Present treatments and care fail because they do not extrapolate into the lifelong (eating and activity) habit changes required. Surgery is costly with short and long term risks and consequences depending on the procedure and the patient. Eating the proper amounts of the right types of foods is key to addressing the problem of obesity.

The following are examples of existing medical and surgical treatments for obesity:
Example: Medication: BELVIQ
Generic: Lorcaserin
Drug Class: Selective Serotonin Agonist
Schedule IV Approved
Cost: $200-220/month
Benefits: Decreases hunger and appetite Increased satiety
3-5% weight loss/year vs. placebo
Risks: Significant relapse
Significant side-effects vs. modest efficacy so rejected by European Medicine Agency Withdrawal
Medication interaction
Side-effects: psychiatric, neurologic, gastrointestinal, cardiologic, endocrine, and hematologic Metabolic issues
Pregnancy: No
Children: No
Example: Medication: CONTRAVE
Generic: Buproprion/Naltrexone
Drug Class: SSRI Antidepressant/Opioid Antagonist Approved
Cost: $180-210/month
Benefits: Decreases hunger and cravings 5% weight loss/year vs. placebo
Risks: Medication interaction Withdrawal
Metabolic and endocrine abnormalities
Side effects: Neurologic, psychiatric, cardiologic, and gastrointestinal
Pregnancy: No
Children: No
Example: Medication: TOPAMAX
Generic: Topiramate
Drug Class: Neurologic
Psychiatric Not approved
Cost: $10-25/month
Benefits: Decreases hunger and craving Migraines
Pain syndromes Seizures
Risks: Minimal, if any efficacy (vs. placebo), and significant side-effects Interactions including Lithium and alcohol
Side-effects: Psychiatric, neurologic, orthopedic, and pulmonary Allergic reactions
Metabolic and endocrine abnormalities Glaucoma
Pregnancy: No
Children: No
Example; Medication: XENICAL (RX)/ALLI (OTC)
Generic: Orlistat
Drug Class: Lipase inhibitor
Approved
Cost: $180/month
Benefits: Lipase inhibitor decreases fat absorption (most concentrated source of
calories) 5% weight loss (vs. placebo)/6 months
Net loss approximately 500 kcal/day=1 lb./week
Risks: TIO dosing
Severe gastrointestinal distress/dumping Vitamin malabsorption
Many medication interactions due to malabsorption problems Side-effects: Liver, pancreas, gallbladder
Complicates and exacerbates gastrointestinal comorbidities
Pregnancy: No
Children: No
Example: Medication: ZONEGRAN
Generic: Zonisamide
Drug Class: Anticonvulsant
Carbonic anhydride inhibitor Not approved
Cost: $30/month
Benefits: 5%-10% weight loss/year vs. placebo Satiety
Decreases hunger and cravings
Indications for seizures, migraines, tremors, neuropathy, and psychiatric disorders
Risks: Large number of psychiatric and neurologic side-effects Significant metabolic issues
Hypersensitivity and allergic reactions Withdrawal
Diuresis
Multiple drug interactions Gastrointestinal symptoms
Pregnancy: No
Children: No
Example: Medication: SAXENDA/VICTOZA
Generic: Liraglutide
Drug Class: GLP-1 receptor agonist Approved
Cost: $20-30/day
Benefits: Satiety
Decreased hunger Diabetes/bloodglucose control
5% weight loss for 60% patients/one year 10% weight loss for 30% patients/one year (vs. 5% weight loss for 30% patients/one year placebo)
Risks: Daily injections
Slows gastric emptying
Nausea and many gastrointestinal side-effects Severe interactions with alcohol
Pancreatitis, renal insufficiency, gallbladder disease, psychiatric issues, cardiologic symptoms Thyroid and breast cancers
Pregnancy: No
Children: No
Example: Medication: QSYMIA
Generic: Phentermine and Topamax
Drug Class: Stimulant/anorectic and seizure Schedule IV
Approved
Cost: $200-220/month
Benefits: Decreases hunger appetite Satiety
16-33 lb./year weight loss (vs. 6 lb./year placebo)
Risks: Significant side-effects Topamax Significant side-effects Phentermine
Information waiver must be signed due to side-effects Tolerance
Withdrawal
Hypersensitivity and allergic reactions Medication interactions
Side-effects: psychiatric, neurologic, cardiac and hypertension, gastrointestinal and glaucoma Metabolic and endocrine dysfunction
Pregnant: No
Children: No
Example: Medications: ZOLOFT, PROZAC, AND WELLBUTRIN
Generic: Sertraline, Fluoxetine, and Bupropion
Drug Class: SSRI Antidepressants
Not approved
Cost: $10-40/month
Benefits: May treat appetite and hunger
May treat psychogenic eating disorders (ie. Overeating due to depression)
Risks: No evidence of weight loss No drug trials for weight loss Possible weight gain
Have been used inappropriately in substitution for Fenflouramine (PONDIMIN) Drug interactions
Withdrawal
Psychologic and neurologic side-effects
Pregnancy: NO
Children: No
Example: Medications: ADIPEX, TENUATE, DIDREX, ADIPOST, and BONTRIL
Generic: Phentermine, Diethylpropion, Benzphetamine, and Phendimetrazine
Drug Class: Sympathomimetic
Schedule Ill and IV Approved for short-term use
Cost: $20-40/month
Benefits: Stimulant
Satiety
Decreased hunger and appetite
50% patients have 10% weight loss/12 weeks 80% patients have 5% weight loss/12 weeks
Average 10% weight loss/one year (vs. 3% placebo)
Risks: Addicting
Withdrawal
Medication interactions
Metabolic and endocrine dysfunction
Side-effects: Cardiac, neurologic, psychologic, gastrointestinal, also hematopoietic Also: insomnia, palpitations, and tremors
Most patients rapidly plateau and then regain weight
Does not support lifestyle changes (enables dysfunctional lifestyle)
Pregnancy: No
Children: No
Example: Procedure: SLEEVE GASTRECTOMY
Cost: $10-26K
Description: Removes 80% of stomach
Results: Low post-op/surgical maintenance No foreign body
Modest surgery time Decreases hunger
Decreases Ghrelin, a “hunger hormone”
Approximately 60% weight loss/first year-maintained through 5 years
Risks: Risks of surgery and anesthesia General gastrointestinal complaints Malnutrition and malabsorption
Plateau of weight loss after 6 months—1 year Hospitalization
Nonreversible Potential sever risks Postoperative diet
Pregnancy: No
Children: No
Example: Procedure: “LAP-BAND SYSTEM”-ADJUSTABLE GASTRIC BANDING
Cost: $9-18K
Description: Laparoscopic or open placement of adjustable restricting gastric band
Results: Forces satiety
Approximately 50% weight loss over 2-3 years (3 months—20%)
(6 months—30%)
(12 months—40%)
(2 years—50%)
Risks: Most weight loss plateaus at 2 years Obstructive
Greatly restricts diet Foreign body and infection
Enabling—does not address abnormal eating behavior Regain weight after removal
Slower weight loss than other surgeries Malnutrition
Costs of removal or revision
Esophago/Gastro/Intestinal symptoms: pain, nausea, vomiting, reflux and esophagitis, slippage
Pregnancy: No
Children: No
Example: Procedure: “ORBERA”—INTRAGASTRIC BALLOON
Cost: $6-8K
Description: Endoscopic placement of intragastric balloon under anesthesia
Results: 20 lb. weight loss/6 months Noninvasive
300% more effective than diet and exercise Easily reversible
20 years of experience—220,000 placed worldwide
Risks: Procedural/placement Obstruction symptoms
No long term benefit—regain weight
Multiple nonspecific gastrointestinal complaints Does not directly address abnormal eating behaviors
Contraindicated with most gastrointestinal conditions Removal
Pregnancy: No
Children: No
Example: Procedure: “ASPIRE ASSIST”—GASTROSTOMY WITH GASTRIC DRAINAGE
Cost: $8-13K
Description: Surgical gastrostomy with application of drainage/evacuation device
Results: Drains 30% postprandial gastric contents
Same procedure as Gastrostomy Placement—many years of experience Newest approved procedure
Reversible
300% more effective than diet and exercise
Risks: Minor surgery
Abdominal tube/hole with hardware/mechanism
Does not address abnormal eating behavior (enables—essentially is medical bulimia) Nonspecific gastrointestinal complaints
Open wound/hole in abdomen No benefit after discontinued
Pregnancy: No
Children: No
Example: Procedure: “VBLOC”—VAGAL NERVE BLOCKADE
Cost: $20-30K
Description: Implanted pulse generator in chest, lead wires to vagus nerve, and controller/charger/transmitter—Enteromedics
Results: 9% weight loss/12 months Satiety
Less hunger Reversible Modestly invasive
Risks: Surgical and anesthesia
Marginally more effective than medical (diet and exercise) Foreign body
Nonspecific gastrointestinal complaints Does not address abnormal eating behavior Regain weight after discontinued
Bloating symptoms with slowed digestion New—unknown long term risks or side-effects
Pregnancy: No
Children: No
Example: Procedure: ROUX-EN-Y GASTRIC BYPASS
Cost: $15-55K
Description: Open or Laparoscopic anastomoses gastric pouch to jejunum and the gastric remnant/duodenum to the distal jejunum
Results: “Gold Standard” of bariatric surgery
60-80% excess weight loss/18 months 50-55% excess weight loss/10 years Satiety
Malabsorption
Many years of experience performing procedure and then treating/following postoperatively
Risks: Surgery and Anesthesia with relatively long procedure Hospitalization
Malabsorption and malnutrition
Postoperative surgical risks with extensive procedure Nonspecific gastrointestinal complaints
Diet restriction Permanent
Eventual weight gain as tolerance develops and eating disorders return
Pregnancy: No
Children: No
Example: Procedure: BILIOPANCREATIC DIVERSION WITH DUODENAL SWITCH
Cost: $24-32K
Description: Open or laparoscopic partial gastrectomy, cholecystectomy, partial duodenectomy, anastomosis of stomach to ileum and duodenum to distal ileum.
Results: Most weight loss of any procedure: 30% excess weight loss/3 months
45% excess weight loss/6 months
65% excess weight loss/12 months 70% weight loss long term
Satiety Malabsorption
Less ulcer and dumping risks as compared to gastric bypass
Risks: Major/extensive surgery and anesthesia Short and long term surgical complications Malabsorption and malnutrition Nonspecific gastrointestinal symptoms Highest complication rate
Permanent Hospitalization
Prolonged recovery: 4-8 weeks
Pregnancy: No
Children: No
WEIGHT LOST—CALORIC EQUIVALENT

| Pounds | Calories (kcal) | kcal/day/6 months | kcal/day/12 months |
|---|---|---|---|
| 5 | 17,500 | 100 | 50 |
| 10 | 35,000 | 200 | 100 |
| 15 | 52,500 | 300 | 150 |
| 20 | 70,000 | 400 | 200 |
| 25 | 87,500 | 500 | 250 |
| 30 | 105,000 | 600 | 300 |
| 35 | 122,500 | 700 | 350 |
| 40 | 140,000 | 800 | 400 |
| 45 | 157,500 | 900 | 450 |
| 50 | 175,000 | 1,000 | 500 |
| 55 | 192,500 | 1,100 | 550 |
| 60 | 210,000 | 1,200 | 600 |

There is accordingly a long-felt need for a safe and effective drug therapy that will suppress appetite in order to prevent, mitigate, treat and/or ameliorate obesity.

### SUMMARY

In certain embodiments, the present invention provides a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

In certain embodiments, the histamine antagonist is azelastine or a pharmaceutically acceptable salt of azelastine or olopatadine or a pharmaceutically acceptable salt of olopatadine.

In certain embodiments, the histamine antagonist is azelastine or a pharmaceutically acceptable salt of azelastine.

In certain embodiments, the histamine antagonist is a pharmaceutically acceptable salt of azelastine.

In certain embodiments, the pharmaceutically acceptable salt of azelastine is azelastine hydrochloride.

In certain embodiments, the local anesthetic is lidocaine or a pharmaceutically acceptable salt of lidocaine.

In certain embodiments, the pharmaceutically acceptable salt of lidocaine is lidocaine hydrochloride.

In certain embodiments, the temperature greater than ambient temperature is a temperature obtained in a nasal cavity of the subject.

In certain embodiments, the temperature greater than ambient temperature is a temperature from 34 degrees Celsius to 40 degrees Celsius.

In certain embodiments, the temperature greater than ambient temperature is 34 degrees Celsius.

In certain embodiments, the temperature greater than ambient temperature is 34.5 degrees Celsius.

In certain embodiments, the temperature greater than ambient temperature is 37 degrees Celsius.

In certain embodiments, the present invention provides a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

In certain embodiments, the histamine antagonist is azelastine or a pharmaceutically acceptable salt of azelastine or olopatadine or a pharmaceutically acceptable salt of olopatadine.

In certain embodiments, the histamine antagonist is azelastine or a pharmaceutically acceptable salt of azelastine.

In certain embodiments, the histamine antagonist is a pharmaceutically acceptable salt of azelastine.

In certain embodiments, the pharmaceutically acceptable salt of azelastine is azelastine hydrochloride.

In certain embodiments, the local anesthetic is lidocaine or a pharmaceutically acceptable salt of lidocaine.

In certain embodiments, the pharmaceutically acceptable salt of lidocaine is lidocaine hydrochloride.

In certain embodiments, the temperature greater than ambient temperature is a temperature obtained in a nasal cavity of the subject.

In certain embodiments, the temperature greater than ambient temperature is a temperature from 34 degrees Celsius to 40 degrees Celsius.

In certain embodiments, the temperature greater than ambient temperature is 34 degrees Celsius.

In certain embodiments, the temperature greater than ambient temperature is 34.5 degrees Celsius.

In certain embodiments, the temperature greater than ambient temperature is 37 degrees Celsius.

In certain embodiments, the present invention provides a process for inducing anosmia in a human subject, the process comprising administering to the subject a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

In certain embodiments, the present invention provides a process for inducing anosmia in a human subject, the process comprising administering to the subject a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

In certain embodiments, the present invention provides a process for treating obesity in a human subject, the process comprising administering to the subject a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

In certain embodiments, the present invention provides a process for treating obesity in a human subject, the process comprising administering to the subject a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

In certain embodiments, the present invention provides a process for suppressing appetite in a human subject, the process comprising administering to the subject a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

In certain embodiments, the present invention provides a process for suppressing appetite in a human subject, the process comprising administering to the subject a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

In certain embodiments, the present invention provides a product comprising: an aerosolizing pump suitable for intranasal administration of a pharmaceutical liquid; and a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

In certain embodiments, the present invention provides a product comprising: an aerosolizing pump suitable for intranasal administration of a pharmaceutical liquid; and a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

### DESCRIPTION OF FIGURE

FIG. 1. Two views of container-closure systems suitable for intranasal delivery of a formulation.

### DESCRIPTION

In connection with the present disclosure it has been conceived inter alia that a formulation comprising a local anesthetic, a drying agent such as an amphetamine, and one or more appropriate excipients can cause suppression of appetite in a human subject to whom the formulation is administered intranasally, for example, when an amount of the formulation is contacted with the superior inner aspect of the nasal mucosa. Such a formulation has been reduced to practice. It is to be desired that the formulation persist in contact with a target anatomical site for conferral of therapeutic benefit. For this reason, the one or more appropriate excipients may be selected so as to enhance persistence of the formulation in contact with a target anatomical site. In addition, the one or more appropriate excipients may be selected to as to enhance stability of the formulation. In addition, the one or more appropriate excipients may be selected so that overall properties of the formulation make the formulation appropriate for human use, with respect to pH, the ability to be sprayed effectively so as to contact a target anatomical site, and so forth. Thus, routine experimentation would have been inadequate to yield a formulation such as is the subject of the present disclosure.

A composition and/or process such as is described in various embodiments herein now will be described more fully hereinafter. A composition and/or process such as is described in various embodiments herein may, however, be embodied in many different forms and should not be construed as limited to the embodiments set forth herein; rather, these embodiments are provided so that this disclosure will be thorough and complete, and will fully convey the scope of a composition and/or process such as is described in various embodiments herein to those skilled in the art. As used in this specification and the claims, the singular forms “a,” “an,” and “the” include plural referents unless the context clearly dictates otherwise. When used in this specification and the claims as an adverb rather than a preposition, “about” means “approximately” and comprises the stated value and every non-negative value within 10% of that value; in other words, “about 100%” includes 90% and 110% and every value in between. Unless stated otherwise, every range or interval includes both endpoints and every value in between.

Certain formulations as described herein included Azelastine Hydrochloride (AZ), Olopatadine Hydrochloride (OL) and/or Lidocaine Hydrochloride (LD). An informal stability study was conducted for six prototype formulations at the 40° C./75% RH accelerated and 25° C./60% RH real time storage conditions for up to 3 months.

Formulations were prepared. The compositions of these formulations are provided in Tables 1-5. These six formulations were prepared at 500 g batch size, and used in a prestability study. Up to 3-month data were acquired from the prestability study.

TABLE 1
Formulas of Prototype Formulation 1 and Prototype Formulation 2

| | Quantity | | | | | | | |
|---|---|---|---|---|---|---|---|---|
| | Prototype Formulation 1 | | | | Prototype Formulation 2 | | | |
| | | | w/w % | w/w % in | | | w/w % | w/w % in |
| Ingredients | Quantity | Unit | in Part A | Formulation | Quantity | Unit | in Part A | Formulation |
| Part A (Vehicle) | | | | | | | | |
| Purified Water, | 298.25 | g | 59.37 | 58.38 | 298.25 | g | 59.37 | 58.68 |
| USP | | | | | | | | |
| Pectin (Low | 5.06 | g | 1.01 | 0.99 | 5.06 | g | 1.01 | 1.00 |
| Methoxy, Non- | | | | | | | | |
| amidated) | | | | | | | | |
| Methocel A4M | 62.09 | g | 12.36 | 12.15 | 62.09 | g | 12.36 | 12.22 |
| Solution (5% w/w) | | | | | | | | |
| Sodium Citrate | 125.18 | g | 24.92 | 24.50 | 125.18 | g | 24.92 | 24.63 |
| Solution (0.1 M/ | | | | | | | | |
| pH 6.0) | | | | | | | | |
| Hydroxypropyl | 0.51 | g | 0.10 | 0.10 | 0.51 | g | 0.10 | 0.10 |
| Methylcellulose | | | | | | | | |
| Sodium Chloride | 0.75 | g | 0.15 | 0.15 | 0.75 | g | 0.15 | 0.15 |
| Sorbitol Solution | 5.03 | g | 1.00 | 0.98 | 5.03 | g | 1.00 | 0.99 |
| (70%) | | | | | | | | |
| Propylene Glycol | 5.02 | g | 1.00 | 0.98 | 5.02 | g | 1.00 | 0.99 |
| Benzalkonium | 0.50 | g | 0.10 | 0.10 | 0.50 | g | 0.10 | 0.10 |
| Chloride | | | | | | | | |
| Part A Total | 502.39 | g | 100.00 | 98.33 | 502.39 | g | 100.00 | 98.85 |
| Formulation | | | | | | | | |
| Part A (Vehicle) | 196.67 | g | NA | 98.33 | 197.74 | g | NA | 98.85 |
| Olopatadine HCl | 1.33 | g | NA | 0.67 | N/A | NA | NA | N/A |
| Lidocaine HCl | 2.01 | g | NA | 1.00 | 2.00 | g | NA | 1.00 |
| Azelastine HCl | None | NA | NA | NA | 0.30 | g | NA | 0.15 |
| Formulation Total | 200.01 | g | NA | 100.00 | 200.05 | g | NA | 100.00 |

TABLE 2
Formula of Prototype Formulation # 3

| | Quantity | | | | |
|---|---|---|---|---|---|
| | Prototype 3 | | | | |
| | | | w/w % | w/w % | |
| | | | in | in | w/w % in |
| Ingredients | Quantity | Unit | Part A | Part B | Formulation |

Part A (Vehicle)

| | | | | | |
|---|---|---|---|---|---|
| Purified Water, USP | 87.55 | g | 17.50 | 17.21 | 17.15 |
| 5% Hydrated Pectin | 75.02 | g | 14.99 | 14.74 | 14.70 |
| (CPKelco) | | | | | |
| 10% Hydrated | 80.17 | g | 16.02 | 15.76 | 15.70 |
| Methocel A15 Premium | | | | | |
| LV (Dow) | | | | | |
| 5% Hydrated Methocel | 45.04 | g | 9.00 | 8.85 | 8.82 |
| A4M Premium LV | | | | | |
| (DOW) | | | | | |
| pH 6.0 0.1M Sodium | 125.00 | mL | 24.98 | 24.57 | 24.49 |
| Citrate Solution | | | | | |
| Sorbitol Solution | 45.72 | g | 9.14 | 8.99 | 8.96 |
| (70%) (Spectrum) | | | | | |
| Polyethylene Glycol | 16.65 | g | 3.33 | 3.27 | 3.26 |
| 400 (Spectrum) | | | | | |
| E10M Hydroxypropyl | 0.60 | g | 0.12 | 0.12 | 0.12 |
| Methylcellulose (Dow) | | | | | |
| Sodium Chloride (J T | 2.05 | g | 0.41 | 0.40 | 0.40 |
| Baker) | | | | | |
| Mannitol | 20.75 | g | 4.15 | 4.08 | 4.06 |
| Benzalkonium | 0.51 | g | 0.10 | 0.10 | 0.10 |
| Chloride (Science Lab) | | | | | |
| Butylated | 0.05 | g | 0.01 | 0.01 | 0.01 |
| Hydroxytoluene | | | | | |
| (Spectrum) | | | | | |
| Glycerol Ester of | 0.61 | g | 0.12 | 0.12 | 0.12 |
| Hydrogenated Rosin | | | | | |
| (Newport Industries) | | | | | |
| Oleic Acid (TCI) | 0.61 | g | 0.12 | 0.12 | 0.12 |
| Part A Total | 500.33 | g | 100.00 | 98.33 | 98.01 |

Part B (Formulation
without Flavors)

| | | | | | |
|---|---|---|---|---|---|
| Part A (Vehicle) | 196.74 | g | NA | 98.33 | 98.01 |
| Olopatadine HCl | 1.34 | g | NA | 0.67 | 0.67 |
| Lidocaine HCl | 2.00 | g | NA | 1.00 | 1.00 |
| Formulation without | 200.08 | g | NA | 100.00 | 99.67 |
| Flavors Total | | | | | |
| Formulation | | | | | |
| Formulation without | 19.06 | g | NA | NA | 99.67 |
| Flavors | | | | | |
| Lavender Flavor | 0.03 | g | NA | NA | 0.17 |
| WONF, Natural, | | | | | |
| Rebertet | | | | | |
| Lemon Flavor WONF, | 0.03 | g | NA | NA | 0.16 |
| Natural, Rebertet | | | | | |
| Formulation Total | 19.12 | g | NA | NA | 100.00 |

TABLE 3
Formula of Prototype Formulation #4

| | Quantity | | | | |
|---|---|---|---|---|---|
| | Prototype 4 | | | | |
| | | | w/w % | w/w % | |
| | | | in | in | w/w % in |
| Ingredients | Quantity | Unit | Part A | Part B | Formulation |

Part A (Vehicle)

| | | | | | |
|---|---|---|---|---|---|
| Purified Water, USP | 87.55 | g | 17.50 | 17.30 | 17.24 |
| 5% Hydrated Pectin | 75.02 | g | 14.99 | 14.82 | 14.77 |
| (CPKelco) | | | | | |
| 10% Hydrated Methocel | 80.17 | g | 16.02 | 15.84 | 15.79 |
| A15 Premium LV (Dow) | | | | | |
| 5% Hydrated Methocel | 45.04 | g | 9.00 | 8.90 | 8.87 |
| A4M Premium LV | | | | | |
| (DOW) | | | | | |
| pH 6.0 0.1M Sodium | 125.00 | mL | 24.98 | 24.70 | 24.61 |
| Citrate Solution | | | | | |
| Sorbitol Solution (70%) | 45.72 | g | 9.14 | 9.03 | 9.00 |
| (Spectrum) | | | | | |
| Polyethylene Glycol 400 | 16.65 | g | 3.33 | 3.29 | 3.28 |
| (Spectrum) | | | | | |
| E10M Hydroxypropyl | 0.60 | g | 0.12 | 0.12 | 0.12 |
| Methylcellulose (Dow) | | | | | |
| Sodium Chloride (J T | 2.05 | g | 0.41 | 0.40 | 0.40 |
| Baker) | | | | | |
| Mannitol | 20.75 | g | 4.15 | 4.10 | 4.09 |
| Benzalkonium Chloride | 0.51 | g | 0.10 | 0.10 | 0.10 |
| (Science Lab) | | | | | |
| Butylated | 0.05 | g | 0.01 | 0.01 | 0.01 |
| Hydroxytoluene | | | | | |
| (Spectrum) | | | | | |
| Glycerol Ester of | 0.61 | g | 0.12 | 0.12 | 0.12 |
| Hydrogenated Rosin | | | | | |
| (Newport Industries) | | | | | |
| Oleic Acid (TCI) | 0.61 | g | 0.12 | 0.12 | 0.12 |
| Part A Total | 500.33 | g | 100.00 | 98.85 | 98.52 |

Part B (Formulation
without Flavors)

| | | | | | |
|---|---|---|---|---|---|
| Part A (Vehicle) | 197.70 | g | NA | 98.85 | 98.52 |
| Lidocaine HCl | 2.00 | g | NA | 1.00 | 1.00 |
| Azelastine HCl | 0.30 | g | NA | 0.15 | 0.15 |
| Formulation without | 200.00 | g | NA | 100.00 | 99.67 |
| Flavors Total | | | | | |
| Formulation | | | | | |
| Formulation without | 19.06 | g | NA | NA | 99.67 |
| Flavors | | | | | |
| Lavender Flavor WONF, | 0.03 | g | NA | NA | 0.17 |
| Natural, Rebertet | | | | | |
| Lemon Flavor WONF, | 0.03 | g | NA | NA | 0.16 |
| Natural, Rebertet | | | | | |
| Formulation Total | 19.12 | g | NA | NA | 100.00 |

TABLE 4
Formula of Prototype Formulation #5

| | Quantity | | | | |
|---|---|---|---|---|---|
| | Prototype 5 | | | | |
| | | | w/w % in | w/w % in | w/w % in |
| Ingredients | Quantity | Unit | Part A | Part B | Formulation |

Part A (Vehicle)

| | | | | | |
|---|---|---|---|---|---|
| Purified Water, USP | 261 | g | 52.18 | 52.08 | 51.21 |
| Microcrystalline | 10 | g | 2.00 | 2.00 | 1.96 |
| Cellulose/CMC Combo (FMC | | | | | |
| Avicel RC-591) | | | | | |
| Methylcellulose (5% | 60 | g | 12.00 | 11.97 | 11.77 |
| Hydrated Methocel A4M | | | | | |
| Premium LV, | | | | | |
| DOW) | | | | | |
| pH 6.0 0.1M Sodium | 125 | g | 24.99 | 24.94 | 24.53 |
| Citrate Solution | | | | | |
| Polysorbate 80 (Croda) | 12.5 | g | 2.50 | 2.49 | 2.45 |
| Hydroxypropyl | 0.6 | g | 0.12 | 0.12 | 0.12 |
| Methylcellulose (E10M, Dow) | | | | | |
| Glycerin (Spectrum) | 12.5 | g | 2.50 | 2.49 | 2.45 |
| Propylene Glycol | 12.5 | g | 2.50 | 2.49 | 2.45 |
| (Spectrum) | | | | | |
| Butylated | 0.05 | g | 0.01 | 0.01 | 0.01 |
| Hydroxytoluene (Spectrum) | | | | | |
| Oleic Acid (TCI) | 2.5 | g | 0.50 | 0.50 | 0.49 |
| Glycerol Ester of | 1.5 | g | 0.30 | 0.30 | 0.29 |
| Hydrogenated Rosin | | | | | |
| (Newport Industries) | | | | | |
| Benzyl Alcohol (Sigma | 2 | g | 0.40 | 0.40 | 0.39 |
| Aldrich) | | | | | |
| Part A Total | 500.15 | g | 100.00 | 99.80 | 98.14 |

Part B (Vehicle with
Flavors)

| | | | | | |
|---|---|---|---|---|---|
| Part A (Vehicle) | 500 | g | NA | 99.80 | 98.14 |
| Carvone Laevo FCC | 0.750 | g | NA | 0.15 | 0.15 |
| (Vigon) | | | | | |
| Methyl Salicylate (Flavor | 0.250 | g | NA | 0.05 | 0.05 |
| Sciences) | | | | | |
| Part B Total | 501.00 | g | NA | 100.00 | 98.33 |
| Formulation | | | | | |
| Part B | 196.67 | g | NA | NA | 98.33 |
| Olopatadine HCl | 1.3301 | g | NA | NA | 0.67 |
| Lidocaine HCl | 2.0047 | g | NA | NA | 1.00 |
| Formulation Total | 200.01 | g | NA | NA | 100.00 |

TABLE 5
Formula of Prototype Formulation #6

| | Prototype 6 | | | | |
|---|---|---|---|---|---|
| | | | w/w % | w/w % | |
| | | | in | in | w/w % in |
| Ingredients | Quantity | Unit | Part A | Part B | Formulation |

Part A (Vehicle)

| | | | | | |
|---|---|---|---|---|---|
| Purified Water, USP | 261.00 | g | 52.18 | 52.08 | 51.48 |
| Microcrystalline | 10.00 | g | 2.00 | 2.00 | 1.97 |
| Cellulose/CMC Combo | | | | | |
| (FMC Avicel RC-591) | | | | | |
| Methylcellulose (5% | 60.00 | g | 12.00 | 11.97 | 11.83 |
| Hydrated Methocel A4M | | | | | |
| Premium LV, DOW) | | | | | |
| pH 6.0 0.1M Sodium | 125.00 | g | 24.99 | 24.94 | 24.66 |
| Citrate Solution | | | | | |
| Polysorbate 80 (Croda) | 12.50 | g | 2.50 | 2.49 | 2.47 |
| Hydroxypropyl | 0.60 | g | 0.12 | 0.12 | 0.12 |
| Methylcellulose (E10M, | | | | | |
| Dow) | | | | | |
| Glycerin (Spectrum) | 12.50 | g | 2.50 | 2.49 | 2.47 |
| Propylene Glycol | 12.50 | g | 2.50 | 2.49 | 2.47 |
| (Spectrum) | | | | | |
| Butylated Hydroxytoluene | 0.05 | g | 0.01 | 0.01 | 0.01 |
| (Spectrum) | | | | | |
| Oleic Acid (TCI) | 2.50 | g | 0.50 | 0.50 | 0.49 |
| Glycerol Ester of | 1.50 | g | 0.30 | 0.30 | 0.30 |
| Hydrogenated Rosin | | | | | |
| (Newport Industries) | | | | | |
| Benzyl Alcohol (Sigma | 2.00 | g | 0.40 | 0.40 | 0.39 |
| Aldrich) | | | | | |
| Part A Total | 500.15 | g | 100.00 | 99.80 | 98.65 |

Part B (Vehicle with
Flavors)

| | | | | | |
|---|---|---|---|---|---|
| Part A (Vehicle) | 500.00 | g | NA | 99.80 | 98.65 |
| Carvone Laevo FCC | 0.75 | g | NA | 0.15 | 0.15 |
| (Vigon) | | | | | |
| Methyl Salicylate (Flavor | 0.25 | g | NA | 0.05 | 0.05 |
| Sciences) | | | | | |
| Part B Total | 501.00 | g | NA | 100.00 | 98.85 |
| Formulation | | | | | |
| Part B | 197.72 | g | NA | NA | 98.85 |
| Lidocaine HCl | 2.00 | g | NA | NA | 1.00 |
| Azelastine HCl | 0.30 | g | NA | NA | 0.15 |
| Formulation Total | 200.02 | g | NA | NA | 100.00 |

Samples of Prototype Formulations 1-6 were packed in glass scintillation vials with caps and HDPE (High Density Polyethylene) bottles with pump and caps, and stored in stability chambers of accelerated 40° C./75% RH condition, 25° C./60% RH real time condition and at 60° C. The samples were tested at different pull time points for the following quality attributes of the formulation: Assays of APIs (LD, OL, and AZ); Appearance (Phase separation, aggregate formation, color, opaqueness and bubbles); Viscosity; pH; Deliverable Weight (from bottles with pump and cap); Weight Change (from bottle with pump and cap).

Results of API Assay at 40° C./75% RH: The Assays of LD, OL and AZ trended down approximately 5% or less, depending on the individual prototype formulations. By examining the Assay testing results, it was noticed that the LD Assay in Prototypes 1, 3 and 5 and the AZ Assay in Prototype 6 decreased significantly (4.2-5.2%), while all other Assays did not decrease significantly (<3%) after 3 months of storage at the 40° C./75% RH condition.

Results of API Assay at 25° C./60% RH: The Assays of LD, OL and AZ trended down approximately 5% or less, depending on the individual prototype formulations, except the OL Assay of Prototype 1 which trended down dramatically by ˜43%. This dramatic decrease in the OL Assay in Prototype 1 was accompanied by the phase separation and aggregate formation observed in the samples which was believed to cause precipitation of OL from the solution phase of the formulation. By examining the Assay testing results, it was noticed that the LD Assay in Prototypes 1, 3 and 5 decreased significantly (4.0-5.1%), while all other Assays did not decrease significantly (<3%) after 3 months of storage at the 25° C./60% RH condition, again, except the OL Assay of Prototype 1 which decreased by ˜43% which was believed to be caused by precipitation of the OL from the solution phase.

Results of API Assay at 60° C. after 1 Week: The changes in Assay for any of the APIs in all prototype formulations were insignificant. Based on these testing results, testing for all prototype formulations was continued for later testing time points at the 25° C./60% RH and 40° C./75% RH conditions.

TABLE 6
Assay Testing Results (% label claim of API) of Prototype
Formulations

| Time | | Prototype 1 | | Prototype 2 | |
|---|---|---|---|---|---|
| (Month) | Condition | LD | OL | LD | AZ |
| 0 | Ambient | 102.7 | 101.6 | 99.1 | 102.3 |
| 0.23 | 60° C. | 101.8 | 101.4 | 101.2 | 102.5 |
| 0.47 | 40° C./75% RH | 102.1 | 101.5 | 101.6 | 102.9 |
| 1 | 40° C./75% RH | 100.4 | 100.5 | 100.5 | 102.2 |
| 2 | 40° C./75% RH | 100.2 | 101.6 | 100.0 | 101.3 |
| 3 | 40° C./75% RH | 98.5 | 99.3 | 98.4 | 100.9 |
| 1 | 25° C./60% RH | 100.6 | 86.9 | 100.8 | 102.3 |
| 3 | 25° C./60% RH | 98.7 | 58.6 | 98.3 | 100.6 |
| Decrease | 25° C./60% RH | −4.0 | −43.0 | −0.8 | −1.7 |
| at 3 MO | | | | | |
| Decrease | 40° C./75% RH | −4.2 | −2.3 | −0.7 | −1.4 |
| at 3 MO | | | | | |

TABLE 7
Assay Testing Results (% label claim of API) of Prototype
Formulations

| Time | | Prototype 3 | | Prototype 4 | |
|---|---|---|---|---|---|
| (Month) | Condition | LD | OL | LD | AZ |
| 0 | Ambient | 103.0 | 102.0 | 100.8 | 104.2 |
| 0.23 | 60° C. | 101.7 | 102.4 | 101.6 | 102.7 |
| 0.47 | 40° C./75% RH | 100.6 | 102.1 | 100.6 | 102.1 |
| 1 | 40° C./75% RH | 100.8 | 99.7 | 101.0 | 104.8 |
| 2 | 40° C./75% RH | 100.0 | 100.3 | 100.3 | 100.8 |
| 3 | 40° C./75% RH | 98.0 | 99.1 | 98.1 | 101.3 |
| 1 | 25° C./60% RH | 101.0 | 101.4 | 100.3 | 101.8 |
| 3 | 25° C./60% RH | 97.9 | 99.7 | 98.4 | 102.0 |
| Decrease | 25° C./60% RH | −5.1 | −2.3 | −2.4 | −2.2 |
| at 3 MO | | | | | |
| Decrease | 40° C./75% RH | −5.0 | −2.9 | −2.7 | −2.9 |
| at 3 MO | | | | | |

TABLE 8
Assay Testing Results (% label claim of API) of Prototype
Formulations

| Time | | Prototype 5 | | Prototype 6 | |
|---|---|---|---|---|---|
| (Month) | Condition | LD | OL | LD | AZ |
| 0 | Ambient | 102.7 | 102.4 | 99.4 | 102.5 |
| 0.23 | 60° C. | 101.4 | 102.7 | 100.9 | 101.0 |
| 0.47 | 40° C./75% RH | 101.0 | 102.3 | 100.4 | 102.5 |
| 1 | 40° C./75% RH | 101.1 | 100.9 | 99.5 | 99.7 |
| 2 | 40° C./75% RH | 98.7 | 101.4 | 99.6 | 100.6 |
| 3 | 40° C./75% RH | 98.4 | 99.4 | 97.4 | 97.3 |
| 1 | 25° C./60% RH | 100.4 | 100.4 | 100.8 | 101.1 |
| 3 | 25° C./60% RH | 97.8 | 100.1 | 98.2 | 99.6 |
| Decrease | 25° C./60% RH | −4.9 | −2.3 | −1.2 | −2.9 |
| at 3 MO | | | | | |
| Decrease | 40° C./75% RH | −4.3 | −3.0 | −2.0 | −5.2 |
| at 3 MO | | | | | |

Results of Phase Separation and Aggregate Formation Examination. 25° C./60% RH Condition: Phase separation and aggregate formation were observed for the 1- and 3-month samples of the LD/OL Prototype Formulation 1. Phase separation and aggregate formation were not observed for any other prototype formulations. The aggregates were white, sphere-shaped and 2-3 mm in size and were observed on the bottom of the sample vials. Corresponding to the observation of the aggregates in these samples, dramatic decreases in OL Assay were observed which suggests the precipitation of OL from the solution of the formulation.

Results of Phase Separation and Aggregate Formation Examination. 40° C./75% RH Condition: Phase separation and aggregate formation were also observed for the LD/OL Prototype Formulation 1 for the 40° C./75% RH samples at all time points after the samples were left in the ambient condition for several days. The aggregates were much smaller than the aggregates observed in the 25° C./75% RH samples.

Results of Phase Separation and Aggregate Formation Examination. 60° C. after 1 Week: phase separation was also observed for the LD/OL prototype formulation although no defined aggregates were observed for the 1-week 60° C. samples.

Based on the results of phase separation and aggregate formation examination, Prototype 1 did not appear to be stable physically, while all other Prototypes appeared to be adequate in terms of physical stability.

Results of Viscosity Testing. 40° C./75% RH Condition: The viscosity of Prototypes 5 and 6 (both of which had high viscosity) trended down dramatically during the 3 months of storage at the 40° C./75% RH accelerated condition; The viscosity of Prototypes 1-4 trended down significantly, although not to the extent as Prototypes 5 and 6, during the 3 months of storage at the 40° C./75% RH condition.

Results of Viscosity Testing. 25° C./60% RH Condition: For the 3 months of storage time at the 25° C./60% RH condition, the viscosity of Prototypes 5 and 6 trended down dramatically; however, the viscosity of Prototypes 3 and 4 did not change significantly, and the viscosity of Prototypes 1 and 2 trended down slightly.

Results of Viscosity Testing. 60° C. Condition: The viscosity of Prototypes 2, 3, 4 and 5 decreased significantly, while the viscosity of Prototype 6 increased significantly, indicating the tendency of change of the viscosity of the prototype formulations which required further testing of all prototypes for longer storage times stored at milder storage conditions. The viscosity of the 60° C./1-week sample for Prototype 1 was not tested because the observation of phase separation and aggregate formation for this Prototype.

TABLE 9
Viscosity Testing Results of Prototype (PT) Formulations

| Time | | Viscosity (cP) | | | | | |
|---|---|---|---|---|---|---|---|
| (Month) | Condition | PT 1 | PT 2 | PT 3 | PT 4 | PT 5 | PT 6 |
| 0 | Ambient | NT | 191.5 | 233.9 | 221.0 | 1800.3 | 1945.7 |
| 1 Week | 60° C. | NT | 119.1 | 196.6 | 165.2 | 1758.0 | 2031.3 |
| 2 Weeks | 40° C./75% RH | NT | 172.0 | 216.1 | 200.7 | 1704.0 | 1816.7 |
| 1 | 40° C./75% RH | 156.5 | 154.1 | 199.8 | 190.0 | 1913.7 | 2187.7 |
| 2 | 40° C./75% RH | 138.7 | 136.2 | 172.2 | 163.1 | 1421.0 | 1922.0 |
| 3 | 40° C./75% RH | 125.9 | 122.8 | 158.0 | 152.2 | 1088.0 | 1295.0 |
| 1 | 25° C./60% RH | 183.9 | 188.9 | 239.5 | 218.4 | 1996.3 | 2045.0 |
| 3 | 25° C./60% RH | 171.0 | 169.9 | 232.5 | 211.9 | 1263.7 | 1226.7 |
| Decrease | 25° C./60% RH | −7% | −11% | −1% | −4% | −30% | −37% |
| at 3 Month | | | | | | | |
| Decrease | 40° C./75% RH | −32% | −36% | −32% | −31% | −40% | −33% |
| at 3 Month | | | | | | | |

Given these viscosity testing results, Prototype 3 and Prototype 4 appeared to be most stable in terms of viscosity followed by Prototypes 1 and 2, and Prototypes 5 and 6 were not stable in terms of viscosity even at the 25° C./60% RH storage condition.

Results of pH Testing. The pH testing results of the prototype formulations are shown in Table 10. At the 40° C./75% RH and 25° C./60% RH conditions, no significant trending was observed for the 3 months of storage for either condition. The slight decreases in pH for the 1-month testing point of Prototype 5 and Prototype 3 at the 40° C./75% RH condition and Prototype 6 at the 25° C./60% RH condition were likely due to experimental variability. No significant changes in pH were observed for the testing results of the 60° C. condition. Based on these pH testing results, it is concluded that the buffer system in these prototype formulations is adequate to maintain the pH of these formulations.

TABLE 10
pH Testing Results of Prototype (PT) Formulations

| Time | | pH | | | | | |
|---|---|---|---|---|---|---|---|
| (Month) | Condition | PT 1 | PT 2 | PT 3 | PT 4 | PT 5 | PT 6 |
| 0 | Ambient | 4.12 | 4.52 | 4.05 | 4.66 | 5.06 | 5.73 |
| 1 Week | 60° C. | 4.21 | 4.59 | 4.20 | 4.64 | 5.07 | 5.77 |
| 2 Weeks | 40° C./75% RH | 4.15 | 4.55 | 4.19 | 4.61 | 5.08 | 5.76 |
| 1 | 40° C./75% RH | 4.17 | 4.58 | 3.76 | 4.63 | 4.84 | 5.76 |
| 2 | 40° C./75% RH | 4.16 | 4.55 | 4.20 | 4.56 | 5.04 | 5.73 |
| 3 | 40° C./75% RH | 4.03 | 4.48 | 4.13 | 4.54 | 4.98 | 5.73 |
| 1 | 25° C./60% RH | 4.17 | 4.54 | 4.18 | 4.47 | 5.08 | 5.31 |
| 3 | 25° C./60% RH | 4.25 | 4.53 | 4.07 | 4.56 | 5.02 | 5.66 |
| Decrease from time | 25° C./60% RH | 0.13 | 0.01 | 0.02 | −0.11 | −0.04 | −0.07 |
| 0 | | | | | | | |
| at 3 Month | | | | | | | |
| Decrease from time | 40° C./75% RH | −0.09 | −0.04 | 0.08 | −0.12 | −0.08 | 0.00 |
| 0 | | | | | | | |
| at 3 Month | | | | | | | |

Results of Deliverable Weight Testing. The samples for the deliverable weight testing of the prototype formulations were packed in plastic bottles with two different types of pumps (Type 1: flat nozzle and Type 2: round nozzle). The duration of the testing was three months. For each time point, three spray tests were performed for Deliverable Weight for each prototype. 40° C./75% RH Condition: No significant trending was observed for all prototypes except Prototype 5 which appeared to trend up by ˜0.01 g and Prototype 2 which trended down by ˜0.01 g from the corresponding initial testing results. However, based on the FDA guidance regarding pump delivery of nasal spray product (the weight of individual sprays within 15% of their target weight and the mean within 10% of their target weight), the testing results of all prototypes are considered acceptable, although Prototype 2 has a relatively higher spread (RSD %) compared to other prototypes. 25° C./60% RH Condition: No significant trending was observed for all prototypes except Prototype 5 which appeared to trend up slightly from initial to 2-week testing point. However, based on the FDA guidance regarding pump delivery of nasal spray product (the weight of individual spray s within 15% of their target weight and the mean within 10% of their target weight), the testing results of all prototypes are considered acceptable, although Prototype 2 has a relatively higher spread (RSD %) compared to other prototypes. Based on the testing results, all prototypes are considered stable in terms of deliverable weight from the two types of pumps included in the study.

Results of Weight Loss Testing. 40° C./75% RH Condition: The average weight loss after 1 month of storage at 40° C./75% RH ranged from 0.25%-0.33% of initial sample weight of 19 g. 25° C./60% RH Condition: The average weight loss after 1 month of storage at 25° C./60% RH ranged from 0.16%-0.19% of initial sample weight of 19 g. Based on the data, it can be estimated the weight loss of the prototype formulations will be approximately 4.8% or less (out of 19 g of the formulation) after 24 months of storage at the 25° C./60% RH condition.

Results of Appearance Examination—Color and Opaqueness. No significant changes were observed for any of the prototype formulations for the storage conditions of 40° C./75% RH and 25° C./60% RH for 3 months and 60° C. for 1 week, except Prototypes 1 and 2. For Prototype 1, the opaqueness of the sample changed from moderate to slightly when compared to the initial sample for the 25° C./60% RH condition after 3 months. For Prototype 2, the opaqueness of the sample changed from slightly to moderate for the 60° C. condition after 1 week.

Results of Appearance Examination—Bubbles. For Prototypes 1 and 2, a large quantity of bubbles were observed after 2 weeks for the samples stored at the 60° C. condition and 40° C./75% RH condition. The nature of these bubbles is not known. For Prototypes 5 and 6, large quantities of bubbles were observed for the initial samples and the nature of these bubbles is not known.

Various formulations were prepared as viscous solutions to be sprayed by a high viscosity nasal pump.

APIs (active pharmaceutical ingredients or “actives”) included in various embodiments of a formulation were, for example, lidocaine HCl, olopatadine HCl and/or azelastine HCl.

Descriptions below pertain to, inter alia, the six prototype formulations described above, and how to make and use such formulations. Excipients associated with favorable performance of a formulation included, for example, pectin, methylcelluloses, blend of microcrystalline cellulose/sodium carboxymethylcellulose, and glycerol ester of hydrogenated rosin.

Pectin: Two types of pectin were tested, both low methylester pectins from CP Kelco. Type 1 was LM-104 AS-Z, a partially aminated pectin. The LM-12 CG-Z pectin appeared to be sensitive to calcium and magnesium ions and had a higher viscosity. Both samples were tested for gelation, by using simulated mucus with concentrations of calcium ions mimicking the actual concentration of mucus. The pectin use levels examined were 0.4-1.2% pectin. Higher concentrations were better at effective in situ gelation.

Hydrating pectin preparations: Pectin stock solutions were prepared for dilution purposes for quickly preparing different concentrations of pectin. A 5% solution was the concentration that was used consistently because pectin tends to form a solid at higher concentrations and is difficult to mix cohesively. Pectin should be the first ingredient added to water to properly hydrate. The water should be heated to 75-80° C. and stirred on a stir plate at a high enough rpm to create a vortex in the solution. Often there was clumping of pectin material, and a homogenizer mixer was used to blend the material uniformly. A digital stirrer can be used in addition, instead of a stir plate. The LM-12 CG-Z type pectin solution has a tan orangish color. The CP Kelco LM-12 CG-Z type pectin showed favorable calcium gelation.

Controlling for pH in pectin formulations: Control of pH was important for pectin stability, which can be maintained between pH 4.0-4.5. Such pH was also found to be a favorable range for pectin gelation. 0.1 M Sodium citrate solutions and 0.1 M citric acid solutions were used as pH adjusters. Sodium citrate/citric acid solutions were prepared to adjust formulations to a final concentration of 0.025 M in the preparations. Pectin in aqueous solution tends to be acidic with a range of 2.5 to about 3.0 pH. A mixture of 0.1 M citric acid/sodium citrate at pH 6.0 was effective for making a formulation of about 4.0 to 4.5 pH because of the acidity of pectin. Sugars have been found to exert a protective effect on pectin as well, and sorbitol, mannitol and dextrose have been mixed with pectin formulations in part for this purpose. Hypermellose or hydroxypropyl methylcellulose (HPMC) may function effectively in a pH range between pH 3.0-11, so addition may take place after the pH has been adjusted with citrate solutions. HPMC also is difficult to hydrate unless wet or dry premixed. Sodium chloride has been found to be a suitable dry mixing ingredient, and glycerin has been found to be a suitable wet premix ingredient for hydrating HPMC.

Methylcellulose A15 LV formulation: The viscosities of various methylcellulose A grades were initially studied. A starting point was to prepare 2% solutions. A15 LV, A4C, and A4M were prepared. A15 LV was found suitable for a relatively lower-viscosity but homogeneous solution. Methylcellulose was cooled to 0 to 5° C. for 20 to 40 minutes for clarity and consistent viscosity. Cold or chilled water was used to hydrate the methylcellulose.

Additives for methylcellulose: A 2% solution of methylcellulose A15C was found to gel at 50° C., so additives were evaluated for lowering gelation temperature, as was increasing methylcellulose concentration. A suitable target gelation temperature was 34.5° C., a temperature that obtains in the nasal cavity. Additives evaluated included sorbitol, mannitol, glycerin, and sodium chloride and other salts. Among additives found to lower gelation temperature while permitting a formulation to remain within a target osmolality range were sorbitol and mannitol.

Osmolality/gelation ratio of methylcellulose: A study was undertaken to test the contribution of osmolality for each individual ingredient in the formulation. Based on individual readings, a change in gelation temperature/osmolality ratio was calculated for each ingredient. Each of sorbitol and mannitol was found suitable for lowering gelation temperature while not contributing greatly to osmolality. Formulations were accordingly prepared which had a gelation temperature in a target range and also an osmolality in a target range.

In situ thermal gelation was tested by taking about 0.1 ml of a formulation and placing it in an HPLC vial. A water bath was then heated to 34.5° C. The HPLC vial was then placed in the water bath for two minutes and then removed from the bath. The capped HPLC vial was then inverted and observed for gelation. If the solution gelled it would stay inverted in the bottom of the HPLC vial.

Microcrystalline cellulose/sodium carboxymethylcellulose (Avicel RC-591): A water dispersible hydrocolloid that can be used for pharmaceutical suspensions and emulsions. Avicel RC-591tends to flocculate out of solutions. Formulations were prepared, and processes for making such formulations were developed, so that this flocculation effect might be limited. Hypermellose (HPMC 2910) methoxyl substitution was found to exert a suspending effect with Avicel RC-591. It was also found that a long stir time at a high rpm helped maintain this excipient suitably in suspension. It was found that heating to 60° C. while stirring provided suitable consistency of solution. It was found that suitability of hydration of Avicel RC-591 was enhanced when Avicel RC-591 was the first ingredient mixed in water.

Multiple Avicel solutions were prepared at 2%, but other preparations ranged from 1-2.5% Avicel. Any of sorbitol, methylcellulose, citrates, sodium chloride, glycerin, and polyethylene glycols were used in various embodiments of a formulation comprising Avicel.

Xanthan gum. Solutions were prepared at a range of 0.3-0.6%, which exhibited fairly high viscosities.

Glycerol ester of hydrogenated rosin (GEHR) is derived from ester resins and is stabilized by hydrogenation. GEHR is poorly soluble in aqueous solutions. Several studies were undertaken to determine what solvent solution would be suitable for solubilizing GEHR. Higher alcohols were found to dissolve GEHR, such as benzyl alcohol and phenethyl alcohol. GEHR was also found to dissolve slowly in oleic acid. However, dissolution of GEHR in a solvent was found not sufficient in every instance to prevent precipitation of GEHR when added into various formulations. Phenethyl alcohol was determined to be suitable in various instances for solubilization.

GEHR was combined with Avicel in various formulations. Several studies were undertaken to dissolve GEHR mixtures. Different types and concentrations of surfactants were tested that included polyethylene glycols, polysorbate 80, propylene glycol, and glycerin. The GEHR/benzyl alcohol mixture was added with a dropper to prevent clumps and keep it in solution with a high stirring speed. GEHR was added as the last ingredient in formulations. The Avicel solutions kept the GEHR in solution somewhat consistently under these conditions.

Methylcellulose A4M: this excipient was determined to function suitably as a viscosity adjuster, as well to assist in providing suitable thermal gelation in various embodiments. It was found to be more viscous and quicker to gel than Methylcellulose A15. Studies were performed to determine the effect of Methylcellulose A4M on viscosity in various formulations. Inclusion of high levels of A4M were found to cause clumping in formulations; inclusion of lower levels of A4M were found suitable for providing suitable gelation and/or viscosity properties.

A manufacturing process was developed for Prototype 4. The manufacturing process was used to prepare a 500 g batch size for the formulation. Manufacturing processes for other prototype formulations were also developed and used to prepare prestability batches. A batch formula for Prototype 4 is provided in Table 11.

TABLE 11
Batch Formula of Prototype Formulation 4 (500 g Batch Size)

| | For 500 g | | |
|---|---|---|---|
| Ingredients | Batch | Unit | % w/w |
| Purified Water, USP | 269.6 | g | 53.924 |
| Pectin (CPKelco) | 3.690 | g | 0.738 |
| Methocel A15 Premium LV (DOW) | 7.890 | g | 1.578 |
| Methocel A4M Premium LV (DOW) | 2.220 | g | 0.444 |
| pH 6.0 0.1M Sodium Citrate Solution | 123.0 | mL | 24.600 |
| Sorbitol Solution (70%) (Spectrum) | 45.000 | g | 9.000 |
| Polyethylene Glycol 400 (Spectrum) | 16.400 | g | 3.280 |
| E10M Hydroxypropyl Methylcellulose | 0.593 | g | 0.119 |
| (DOW) | | | |
| Sodium Chloride (J T Baker) | 2.020 | g | 0.404 |
| Mannitol | 20.400 | g | 4.080 |
| Benzalkonium Chloride (Science Lab) | 0.500 | g | 0.100 |
| Butylated Hydroxytoluene (Spectrum) | 0.050 | g | 0.010 |
| Glycerol Ester of Hydrogenated Rosin | 0.603 | g | 0.121 |
| (Newport Oleic Acid (TCI) | 0.603 | g | 0.121 |
| Lidocaine HCl | 5.000 | g | 1.000 |
| Azelastine HCl | 0.750 | g | 0.150 |
| Lavender Flavor WONF, Natural, Rebertet | 0.840 | g | 0.168 |
| Lemon Flavor WONF, Natural, Rebertet | 0.820 | g | 0.164 |
| Formulation Total | 500.0 | g | 100.000 |

Process for making a batch of formulation. A manufacturing process used to make a prestability batch involved the preparation of several premixes/solutions of a number of ingredients. Procedures for preparation of certain premixes/solutions are described as follows.

Preparation of 5% Pectin Solution: Weighed out and added 10 g Pectin in 190 mL of purified water. The mixture was heated to 80° C. while being mixed using a hand mixer until the Pectin hydrated completely.

Preparation of 10% Methocel A15 LV Solution: Weighed out and added 10 g Methocel A15 LV in 90 mL of purified water and the mixture was mixed using a hand mixer until the Methocel was hydrated completely.

Preparation of 5% Methocel A4M Solution: Weighed out and added 10 g Methocel A4M in 90 mL of purified water and the mixture was mixed using a hand mixer until the Methocel was hydrated completely.

Preparation of 0.1 M pH 6.0 Sodium Citrate Buffer Solution: Measured out 442.5 mL 0.1 M Sodium Citrate solution and 57.5 mL 0.1 M Citric Acid solution, mixed and stirred for approximately 10 min.

Preparation of Solution of GEHR in Oleic Acid: Weighed out 1 g of glycerol ester of hydrogenated rosin (GEHR) and 1 g of Oleic acid, mixed and sonicated repeatedly until GEHR dissolved in the Oleic acid completely.

Process Flow Diagram for Prestability Study Batch (500 g Batch Size)
Process Flow Diagram for Optimized Process for Development Activity (500 g Batch Size)

The manufacturing process of the prestability batch as illustrated in Page 2 of the Process Flow Diagram for Prestability Study Batch (500 g Batch Size) starts with measuring out ˜87.6 mL of USP PW into a 1000 mL tall beaker. Measure out and add 75 g of 5% CP Kelco Pectin solution. Using a magnetic stir bar, stir the solution for five minutes. Measure out and add 80 g of 10% A15 methylcellulose. Allow the solution to stir for five minutes. Measure out and add 45 g of 5% A4M methylcellulose. Stir the solution for 10 minutes. At this point, the solution is extremely viscous. Measure out and add 125 mL pH 6.0 sodium citrate solution to the formulation mixture. Measure out and add 45.7 g of sorbitol. Measure out and add 16.65 g of polyethylene glycol 400 (PEG 400) to lower the viscosity. Stir the solution for ˜10 minutes. Separately mix 0.6 g of HPMC with 2 g of sodium chloride, and add this mixture to the formulation mixture. Stir the solution for ˜30 minutes until all chunks are absent from the solution. Measure out and add 20.75 g of mannitol, and stir the solution for 15 minutes. Measure out 0.5 g of benzalkonium chloride and 0.05 g of butylated hydroxytoluene, and add these excipients to the formulation mixture. Stir the formulation mixture for 10 minutes.

Next, 0.6 g of oleic acid and 0.6 g of glycerol ester of hydrogenated rosin (GEHR) are measured out. The GEHR is added to the oleic acid, and the mix is sonicated for ˜10 minutes. Once the GEHR is fully dissolved, the solution is stirred for an additional 5 minutes. This mixture is added to the formulation mixture, and the formulation mixture is stirred for ˜1 hour to ensure uniform solution before transferring to glass bottles.

Measure out ˜197.7 g of the previously prepared formulation mixture, and using the Scilogex OS20-Pro Laboratory Stirrer, begin stirring the formulation mixture at 300 RPM for ˜10 minutes. Weigh out 2.0 g of Lidocaine HCl and 0.3 g of Azelastine HCl. Very slowly add each API to the formulation mixture, avoiding the side of the beaker and shaft of spindle. Azelastine appears to resist going into solution. Stir the formulation mixture for ˜30 minutes, and after 30 minutes, sonicate the formulation mixture for five minutes. If there are still chunks present in the formulation mixture, alternate between stirring and sonicating the formulation mixture until the chunks disappear. After all the excipients are dissolved, stir the formulation mixture for 30 minutes to ensure content uniformity.

Next, ˜19 g of the formulation mixture with the APIs is weighed out into a scintillation vial. 0.03 g of lavender flavor and 0.03 g of lemon flavor are weighed out and added to the formulation mixture in the scintillation vial to produce the a sample of the final formulation of Prototype 4. This sample of the final formulation of Prototype 4 is vortexed and used for the informal prestability study. Multiple samples are prepared by repeating the step of adding the flavor ingredients and final mixing.

Description of Manufacturing Process for Development. The manufacturing process used to prepare the prestability batch was modified based on several process studies. The optimized process is illustrated in the process flow diagram shown in Process Flow Diagram for Optimized Process for Development Activity (500 g Batch Size). The major modifications are listed below.
Azelastine hydrochloride is added to the formulation container as a second ingredient after the addition of the purified water, in order to ensure complete and easy dissolution of this API. (Note that chemical stability of Azelastine in the formulation prepared by this process has to be assessed given the exposure of Azelastine to the high temperature (80° C.) of the next step when pectin is added and dispersed in the formulation mixture. Another option is to add Azelastine after Pectin is added and dispersed and when the formulation mixture is cooled to room temperature).
Solid Pectin, Methocel A15 LV and Methocel A4M are added directly to the formulation container without being hydrated separately in advance.
The lavender flavor and lemon flavor are dissolved in the PEG 400 first, then the mix of the flavors in PEG 400 is added to the formulation mixture. Note: this step has yet to be tried to confirm these flavor ingredients dissolve in PEG 400 readily.

A process for making Prototype 4 such as is shown in Process Flow Diagram for Optimized Process for Development Activity (500 g Batch Size) is described below:

Measure out 269.6 mL of USP PW into a 1000 mL tall beaker. Using the Scilogex OS20-Pro Laboratory Stirrer, begin stirring the purified water at 250 RPM. Measure out 0.75 g of Azelastine HCl, and add to the purified water. Allow the azelastine to completely dissolve in the water before moving on to the next step. Using a hot plate, begin heating purified water to ˜80° C. Cover the beaker with a low evaporation dissolution vessel lid, and begin stirring at 250 RPM. After reaching ˜80° C., measure out 3.69 g of pectin and slowly add to the formulation mixture, increasing stirring speed to 350 RPM. Allow pectin to completely solubilize. This should take ˜10-20 minutes. Next, remove beaker from hot plate and allow the formulation mixture to cool to room temperature. Before allowing the formulation mixture to cool to room temperature, the stirring spindle should be lifted from the formulation mixture and checked for undissolved material. If material is found, use spatula to remove from spindle and add back to formulation mixture. Continue stirring until all material has been dissolved.

Once the formulation mixture has reached room temperature, measure out 2.22 g of Methocel A4M and 7.89 g of Methocel A15 LV methylcellulose. Slowly add the 2.21 g of Methocel A4M to the formulation mixture being careful to avoid the sides of the beaker and/or spindle shaft. Stir the formulation mixture until the Methocel A4M almost completely dissolves (˜10 minutes), and then slowly add the 7.89 g of Methocel A15 LV. Stir the formulation mixture until precipitates are no longer visible (˜30 minutes). Once again, remove stirring spindle from the formulation mixture and check for undissolved material. If material is found, use a spatula to remove from spindle and add back to formulation mixture. Ensure material is dissolved before proceeding.

After the Methocel ingredients completely dissolve, measure out and add 123.0 mL of pH 6.0 sodium citrate solution (The pH 6.0 sodium citrate solution is comprised of 0.1 M citric acid and 0.1 M sodium citrate which should be made in advance). Measure out 2.02 g of sodium chloride and 0.593 g of hydroxypropyl methylcellulose (HPMC). Prior to adding to the formulation mixture, dry mix the two excipients together; this helps with the dissolution of HPMC. After addition of the dry mix of sodium chloride and HPMC, small chunks will form; wait for these chunks to completely dissolve before further excipient addition. This should take ˜10-15 minutes.

Once all the HPMC has successfully solubilized, measure and add 45.0 g of sorbitol and 20.4 g of mannitol, respectively. Mannitol will not dissolve instantaneously. Allow the formulation mixture to stir until mannitol is completely solubilized. Measure out 16.4 g of polyethylene glycol 400 (PEG 400), and mix the PEG 400 with 0.84 g of the lavender flavor and 0.82 g of the lemon flavor, then add the mix of the PEG 400 and the two flavor ingredients to the formulation mixture. Next, measure out 0.5 g of Benzalkonium chloride and 0.05 g of butylated hydroxytoluene, add to the formulation mixture.

Measure out 0.603 g of glycerol ester of hydrogenated rosin (GEHR) and 0.603 g of oleic acid. (Note: GEHR is not readily soluble in aqueous solutions and is typically to be dissolved prior to addition into the formulation mixture). To dissolve GEHR in oleic acid, add the oleic acid to a scintillation vial, and slowly add the GEHR to the oleic acid in the scintillation vial. Sonicate the mix of GEHR and oleic acid for ˜10 minutes or until the GEHR is fully dissolved. Add the mix of the GEHR and oleic acid to the formulation mixture. Stir for ˜20-30 minutes. Next, measure out 5.00 g of Lidocaine HCl, and add to the formulation mixture to produce the final formulation. Stir the final formulation for ˜1 hour to ensure uniform solution.

Mixing Speed: For the process of this formulation, one important process parameter to control is the mixing speed. For some of the excipients, if the mixing speed is higher than 450 RPM, too much air is introduced into the formulation and the solution becomes very bubbly. However, if the mixing speed is lower than 250 RPM, some of the excipients and APIs will not dissolve completely. This would impact the quality of the drug product.

Temperature: Another important process parameter of the process is the temperature of the formulation mixture during the step of dispersing and hydrating the Pectin. If the formulation mixture is not heated to 80° C., the pectin does not readily dissolve and hydrate. It is also important to make sure that the formulation mixture is allowed to cool to room temperature after the step of dispersing and hydrating the pectin. Adding methylcellulose to a heated solution will cause the methylcellulose to gel in solution. This will compromise the quality of the batch. As discussed in Section 3.4, one option to avoid exposing Azelastine to the high temperatures is to add Azelastine Hydrochloride after dispersing and hydrating pectin and when the temperature of the formulation mixture cools to room temperature. Addition of Dextrose to Vehicle System for Prototype 4. The purpose of this study was to add dextrose to improve the gelation properties of the formulation (i.e., further decrease the incipient gelation temperature) if required in development. The batch of the formulation was prepared in 100 g scale without any active pharmaceutical ingredients. The dextrose was added in the last step with the PEG 400, Benzalkonium chloride and the GEHR/oleic acid mixture. The final formulation solution was allowed to stir for ˜1 hour.

After the solution was thoroughly mixed, ˜0.25 mL of the sample was placed in an HPLC vial. The sample was then placed in a 34.5° C. water bath for ˜3 minutes. The solution gelled when exposed to the heightened temperature. The solution remained gelled when placed upside down for an extended period of time. The addition of dextrose seems to increase the gelation capacity of the formulation. Note that the selected formulation Prototype 4 does not contain Dextrose. Investigation of the impact of dextrose on the stability of the formulation is to be conducted if dextrose is added to the formulation.

Formulation uniformity. A batch (500 g batch size) of the formulation of Prototype 4 was prepared to assess the content uniformity of the APIs in the formulation. The Lidocaine Hydrochloride and Azelastine Hydrochloride were added last to the formulation mixture. Azelastine appeared difficult to dissolve. After thorough mixing, the Azelastine dissolved completely. Samples were taken from the top, middle and bottom of the formulation (two samples from each location), and tested for Assays of Lidocaine and Azelastine. The testing results ranged from 102.5-103.4% label claim for Lidocaine and 102.1-103.9% label claim for Azelastine, indicating the formulation was homogeneous in terms of the active pharmaceutical ingredients.

Study of Process Changes of Prototype 4 Formulation. This study was performed to evaluate several changes of the process and three 500 g batches of Prototype 4 formulation were prepared to confirm the changes. The process changes included the following: (1) Pectin, Methocel A4M and Methocel A15 LV were dispersed and hydrated directly in the batch without preparing their stock solutions in advance; (2) Azelastine Hydrochloride was added to the batch as the second ingredient after Purified Water to facilitate the dissolution of Azelastine Hydrochloride. The dissolution of Azelastine Hydrochloride was difficult and slow as observed during the preparation of the prestability batch when added to the formulation mixture at the later stage of the process. In this study, the Azelastine hydrochloride dissolved into the purified water readily after approximately 5 minutes of stirring. The direct dispersion and hydration of Pectin, Methocel A4M and Methocel A15 LV also yielded successful results. The experience and knowledge from this study were used to define the manufacturing process for development activity.

Dissolution of GEHR in oleic acid. Glycerol ester of hydrogenated rosin (GEHR) may pose a challenge for dissolution in a formulation. For the prestability batches, the GEHR was added to the oleic acid, and the mixture of the GEHR and Oleic acid was sonicated until the GEHR dissolved. The mixture of the GEHR and Oleic acid was then added to the formulation mixture. In this study, different methods (without using sonication) of dissolving GEHR into oleic acid were investigated. The oleic acid was added to a scintillation vial with a small stir bar. The oleic acid was then heated to ˜30° C. Then the GEHR was added to the Oleic acid. The mixture of the GEHR and Oleic acid was stirred for six hours and the GEHR dissolved in the Oleic acid completely. No color change was observed after the solution was heated. Other trials with heating the GEHR and oleic acid produced a color change if the solution was heated above 35° C. If sonication is not a viable technique in the manufacturing environment, stirring at ˜30° C. would be an alternative technique to prepare the solution of the GEHR and Oleic acid.

Process for making Prototypes 1 and 2. A process (500 g batch size) for making formulations of Prototypes 1 and 2 is described as follows:
1. Measure out purified water into a 1000 mL tall beaker. Place beaker on a hot plate and begin heating to ˜80° C. Using Scilogex OS20-Pro Laboratory Stirrer, begin stirring water at 250 RPM.
2. Once purified water has reached ˜80° C., measure out pectin and slowly begin adding to formulation being careful to avoid getting material on sides of beaker and/or spindle shaft. Increase stirring speed to 500 RPM. Allow solution to stir until all pectin has been dissolved, this could take ˜20 minutes.
3. After all pectin has been completely solubilized, cool solution to room temperature.
4. Measure and slowly add 5% A4M methylcellulose solution while increasing the stirring speed to 700 RPM.
NOTE: This hydrated solution has a tendency to stick to spindle shaft and/or sides of beaker. If this happens, may need to stop stirring, remove material using spatula and attempt to add to formulation again
5. Add pH 6.0 sodium citrate buffer and increase stirring speed to 750 RPM. NOTE: pH 6.0 sodium citrate buffer is a solution comprised of 0.1 M citric acid and 0.1 M sodium citrate which should be made prior to addition to formulation.
6. Measure out both sodium chloride and hydroxypropyl methylcellulose (HPMC). Prior to adding to formulation, dry mix the two excipients together, this helps with the dissolution of HPMC. After addition of the mixture, small chunks will form; wait for these to completely dissolve before further excipient addition. This should take ˜10-15 minutes.
7. Measure out and add sorbitol, propylene glycol and benzalkonium chloride while increasing stirring speed to 900 RPM. Allow to stir for ˜1 hr. to ensure the solution is uniform
8. API Addition
   a. Lidocaine & Olopatadine (Prototype 1)
      i. Slow stirring speed to 400 RPM.
      ii. Measure out appropriate amounts of both APIs.
         1) Lidocaine should account for 1% of the solution.
         2) Olopatadine should account for 0.67% of the solution
      iii. Slowly add each API (Lidocaine was added first for stability study sample preparation) being careful to avoid sides of beaker and spindle shaft
      iv. Allow to stir for ˜30 minutes. Ensure that both APIs have been completely dissolved before transferring to glass bottle.
   b. Lidocaine & Azelastine (Prototype 2)
      i. Slow stirring speed to 400 RPM.
      ii. Measure out appropriate amounts of both APIs.
         1) Lidocaine should account for 1% of the solution.
         2) Azelastine should account for 0.1% of the solution.
      iii. Slowly add each API (Lidocaine was added first for stability study sample preparation) being careful to avoid sides of beaker and spindle shaft.
      NOTE: Azelastine does not readily go into solution, may have to sonicate and/or vortex.
      iv. Allow to stir for ˜30 minutes. Assess the extent of solubility of Azelastine. If aggregates are still present, sonicate covered beaker for ˜5 to 10 minutes.
      v. Continue stirring ˜15 minutes before once again assessing the extent of solubility of Azelastine. If there are still undissolved precipitates, repeatedly sonicate and stir until they are no longer visible and transfer to glass bottle.

Process for making Prototypes 5 and 6. A process (500 g batch size) for making formulations of Prototypes 5 and 6 is described as follows:
1. Measure out purified water into a 1000 mL glass beaker. Begin heating to ˜60° C. while stirring using stir plate.
2. Measure out and slowly add Avicel to the heated purified water.
3. While the formulation mixture is being stirred, measure out 5% A4M methylcellulose solution and the pH 6.0 citrate buffer.
NOTE: The pH 6.0 citrate buffer is a solution comprised of 0.1 M citric acid and 0.1 M sodium citrate which should be made prior to addition to the formulation mixture.
4. On a separate stir plate, begin stirring the mixture of the 5% A4M methylcellulose and citrate buffer until a uniform solution is produced.
5. Once Avicel has been completely hydrated, allow to cool to room temperature.
6. After the mixture of Avicel and purified water has cooled to room temperature, add the mixture of the 5% A4M methylcellulose and pH 6.0 citrate buffer and stir until a uniform mixture is formed.
7. Measure out the hydroxypropyl methylcellulose (HPMC) and glycerin. Separately mix the HPMC with glycerin (this significantly aids in the dissolution of HPMC). Add the mixture of HPMC and glycerin to the formulation mixture.
8. Measure out and add the propylene glycol, polysorbate 80 and oleic acid into the formulation mixture, respectively.
9. Measure out glycerol ester of hydrogenated rosin (GEHR) and benzyl alcohol (GEHR is not readily soluble in aqueous solutions and is typically to be dissolved in benzyl alcohol prior to addition into the formulation). To dissolve GEHR in benzyl alcohol, mix in scintillation vial and sonicate for ˜15 min, vortex for ˜1 min and repeat until solution is produced.
10. Prior to adding the solution of GEHR in benzyl alcohol to the formulation mixture, begin mixing the formulation mixture with Scilogex OS20-Pro Laboratory Stirrer at 600 RPM. Add the solution of GEHR in benzyl alcohol dropwise and stir until all solution of GEHR in benzyl alcohol completely dissolves.
11. API Addition
   a. Lidocaine & Olopatadine (Prototype 5)
      i. Begin stirring the formulation mixture with Scilogex OS20-Pro Laboratory stirrer at 300 RPM.
      ii. Measure out appropriate amounts of both APIs
         1. Lidocaine should account for 1% of the solution
         2. Olopatadine should account for 0.67% of the solution
      iii. Slowly add each API (Lidocaine was added first for stability study sample preparation) being careful to avoid sides of beaker and spindle shaft.
      iv. Allow to stir for ˜30 minutes. Ensure that both APIs have been completely dissolved before transferring to a glass bottle.
   b. Lidocaine & Azelastine (Prototype 6)
      i. Begin stirring the formulation solution with Scilogex OS20-Pro Laboratory stirrer at 300 RPM.
      ii. Measure out appropriate amounts of both APIs
         1. Lidocaine should account for 1% of the solution
         2. Azelastine should account for 0.1% of the solution
      iii. Slowly add each API (Lidocaine was added first for stability study sample preparation) being careful to avoid sides of beaker and spindle shaft.
      NOTE: Azelastine does not readily go into solution, may have to sonicate and/or vortex.
      iv. Allow to stir for ˜30 minutes. Assess the extent of solubility of Azelastine. If aggregates are still present, sonicate covered beaker for ˜5 to 10 minutes.
      v. Continue stirring ˜15 minutes before once again assessing the extent of solubility of Azelastine. If there are still undissolved precipitates, repeatedly sonicate and stir until they are no longer visible and transfer the final formulation to a glass bottle.

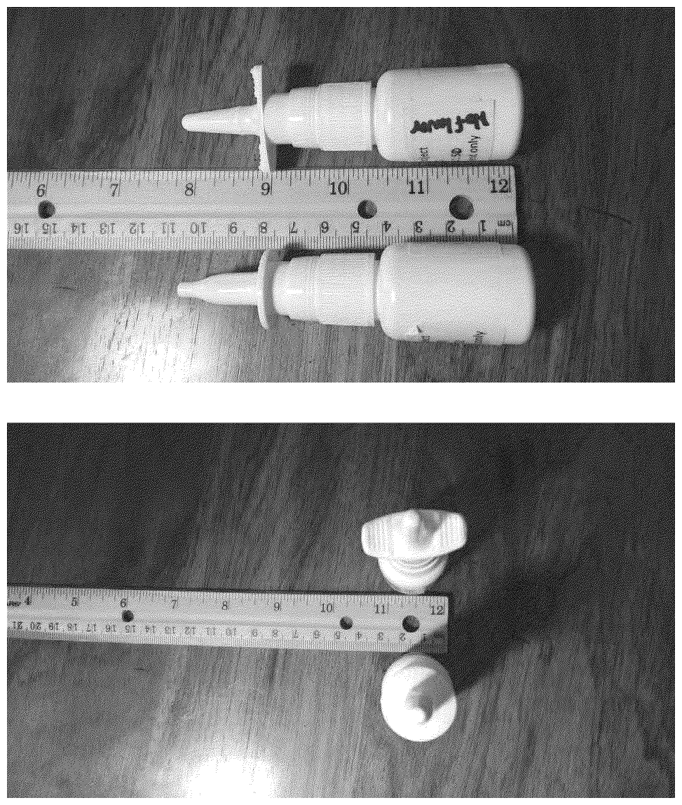

Nose spray pump/aerosolizer. A nose spray pump/device such as is known in the pharmaceutical arts is used to directionally spray an amount of a formulation to the top of the interior of the nasal cavity. A depiction of two such nose spray pump/devices is included in this disclosure as FIG. 1. Such directional spray of a formulation delivers an amount (or a volume or a mass) of a formulation to a target anatomical site of a subject and accordingly affects an olfactory nerve of the subject and blocks the subject's sense of smell. Such a pump/aerosolizer aims an amount of a formulation at and delivers an amount of a formulation to a target anatomical site in a superior portion of a subject's nasal cavity and accordingly does not spray an amount of a formulation uniformly throughout the nasal cavity and sinuses.

In an example, a volume of an appetite suppressant composition as disclosed herein is predominantly in a free-flowing liquid state at ambient temperature. In an example, a volume of an appetite suppressant composition as disclosed herein is predominantly in a gel form, that is, in a form of a semirigid colloidal dispersion, at a temperature of a superior portion of a nasal cavity of a human subject. For example, such an ambient temperature may be as high as 30 degrees Celsius. For example, such a temperature of a superior portion of a nasal cavity may be as low as 34 degrees Celsius.

In an example, a volume of an appetite suppressant composition as disclosed herein is sprayed into a nostril of a human subject so as to contact a superior portion of a nasal cavity of the subject. In an example, a majority of the volume that is sprayed remains in the superior portion of the nasal cavity for a period of at least five minutes immediately following the spraying of the volume.

In an example, a mass of an appetite suppressant composition as disclosed herein is sprayed into a nostril of a human subject so as to contact a superior portion of a nasal cavity of the subject. In an example, a majority of the mass that is sprayed remains in the superior portion of the nasal cavity for a period of at least five minutes immediately following the spraying of the mass.

In an example, a volume of an appetite suppressant composition as disclosed herein is sprayed into a nostril of a human subject so as to contact a superior portion of a nasal cavity of the subject. In an example, less than 25% of the volume that is sprayed exits the nostril from the external aperture thereof during a five-minute interval immediately following the spraying of the volume.

In an example, a mass of an appetite suppressant composition as disclosed herein is sprayed into a nostril of a human subject so as to contact a superior portion of a nasal cavity of the subject. In an example, less than 25% of the mass that is sprayed exits the nostril from the external aperture thereof during a five-minute interval immediately following the spraying of the mass.

In an example, a formulation comprises Purified Water, USP, at a concentration of 53.924% w/w. In an example, a formulation comprises Purified Water, USP, at a concentration of from about 10% to about 90% w/w, or from about 20% to about 80% w/w, or from about 30% to about 70% w/w, or from about 40% to about 60% w/w, or from about 45% to about 55% w/w.

In an example, a formulation comprises pectin at a concentration of 0.738% w/w. In an example, a formulation comprises pectin at a concentration of from about 0.1% to about 1.5% w/w, or from about 0.2% to about 1.25% w/w, or from about 0.3% to about 1.1% w/w, or from about 0.4% to about 1% w/w, or from about 0.5% to about 0.9% w/w, or from about 0.6% to about 0.8% w/w.

In an example, a formulation comprises Methocel A15 Premium LV at a concentration of 1.578% w/w. In an example, a formulation comprises Methocel A15 Premium LV at a concentration of from about 0.25% to about 4% w/w, or from about 0.5% to about 3% w/w, or from about 0.75% to about 2.25% w/w, or from about 1% to about 2% w/w, or from about 1.25% to about 1.75% w/w, or from about 1.4% to about 1.6% w/w.

In an example, a formulation comprises Methocel A4M Premium LV at a concentration of 0.444% w/w. In an example, a formulation comprises Methocel A4M Premium LV at a concentration of from about 0.05% to about 1% w/w, or from about 0.1% to about 0.9% w/w, or from about 0.2% to about 0.8% w/w, or from about 0.3% to about 0.7% w/w, or from about 0.4% to about 0.6% w/w.

In an example, a formulation comprises a 0.1 M sodium citrate aqueous solution, pH 6.0, at a concentration of 24.600% w/w. In an example, a formulation comprises a 0.1 M sodium citrate aqueous solution, pH 6.0, at a concentration of from about 10% to about 40% w/w, or from about 15% to about 35% w/w, or from about 20% to about 30% w/w, or from about 22.5% to about 27.5% w/w, or from about 24% to about 25% w/w.

In an example, a formulation comprises a 70% sorbitol aqueous solution at a concentration of 9.000% w/w. In an example, a formulation comprises a 70% sorbitol aqueous solution at a concentration of from about 1% to about 20% w/w, or from about 2% to about 17% w/w, or from about 4% to about 15% w/w, or from about 6% to about 12% w/w, or from about 8% to about 10% w/w.

In an example, a formulation comprises polyethylene glycol 400 (PEG 400) at a concentration of 3.280% w/w. In an example, a formulation comprises polyethylene glycol 400 at a concentration of from about 0.5% to about 10% w/w, or from about 0.75% to about 8% w/w, or from about 1% to about 6% w/w, or from about 2% to about 5% w/w, or from about 3% to about 4% w/w.

In an example, a formulation comprises E10M hydroxypropyl methylcellulose at a concentration of 0.119% w/w. In an example, a formulation comprises E10M hydroxypropyl methylcellulose at a concentration of from about 0.02% to about 0.2% w/w, or from about 0.04% to about 0.18% w/w, or from about 0.05% to about 0.16% w/w, or from about 0.08% to about 0.14% w/w, or from about 0.1% to about 0.13% w/w.

In an example, a formulation comprises sodium chloride at a concentration of 0.404% w/w. In an example, a formulation comprises sodium chloride at a concentration of from about 0.05% to about 2% w/w, or from about 0.1% to about 1.5% w/w, or from about 0.2% to about 1% w/w, or from about 0.3% to about 0.6% w/w, or from about 0.35% to about 0.45% w/w.

In an example, a formulation comprises mannitol at a concentration of 4.080% w/w. In an example, a formulation comprises mannitol at a concentration of from about 0.5% to about 20% w/w, or from about 1% to about 15% w/w, or from about 2% to about 10% w/w, or from about 3% to about 7.5% w/w, or from about 3.5% to about 4.5% w/w.

In an example, a formulation comprises benzalkonium chloride at a concentration of 0.100% w/w. In an example, a formulation comprises benzalkonium chloride at a concentration of from about 0% to about 0.2% w/w, or from about 0.025% to about 0.175% w/w, or from about 0.05% to about 0.15% w/w, or from about 0.075% to about 0.125% w/w.

In an example, a formulation comprises butylated hydroxytoluene at a concentration of 0.010% w/w. In an example, a formulation comprises butylated hydroxytoluene at a concentration of from about 0% to about 0.02% w/w, or from about 0.0025% to about 0.0175% w/w, or from about 0.005% to about 0.015% w/w, or from about 0.0075% to about 0.0125% w/w.

In an example, a formulation comprises glycerol ester of hydrogenated rosin at a concentration of 0.121% w/w. In an example, a formulation comprises glycerol ester of hydrogenated rosin at a concentration of from about 0.02% to about 0.22% w/w, or from about 0.05% to about 0.19% w/w, or from about 0.08% to about 0.16% w/w, or from about 0.1% to about 0.14% w/w.

In an example, a formulation comprises oleic acid at a concentration of 0.121% w/w. In an example, a formulation comprises oleic acid at a concentration of from about 0.02% to about 0.22% w/w, or from about 0.05% to about 0.19% w/w, or from about 0.08% to about 0.16% w/w, or from about 0.1% to about 0.14% w/w.

In an example, a formulation comprises lidocaine hydrochloride at a concentration of 1.000% w/w. In an example, a formulation comprises lidocaine hydrochloride at a concentration of from about 0% to about 2% w/w, or from about 0.25% to about 1.75% w/w, or from about 0.5% to about 1.5% w/w, or from about 0.75% to about 1.25% w/w.

In an example, a formulation comprises azelastine hydrochloride at a concentration of 0.150% w/w. In an example, an formulation comprises azelastine hydrochloride at a concentration of from about 0% to about 0.3% w/w, or from about 0.05% to about 0.25% w/w, or from about 0.1% to about 0.2% w/w.

In an example, a formulation comprises lavender flavor at a concentration of 0.168% w/w. In an example, a formulation comprises lavender flavor at a concentration of from about 0% to about 0.3% w/w, or from about 0.08% to about 0.25% w/w.

In an example, a formulation comprises lemon flavor at a concentration of 0.164% w/w. In an example, a formulation comprises lemon flavor at a concentration of from about 0% to about 0.3% w/w, or from about 0.08% to about 0.25% w/w.

Assays for active pharmaceutical ingredients such as are disclosed herein are described inter alia in the current edition of the United States Pharmacopeia, which is incorporated herein by reference.

A formulation such as is disclosed herein was administered to a human subject to suppress said subject's appetite. The subject self-administered ad libitum a prototype formulation such as is described above. The subject sprayed the formulation into each of his nasal cavities. The formulation adhered to the subject's nasal mucosa in the superior portion of the subject's nasal cavity. The formulation gelled in situ in the superior portion of the nasal cavity and formed a gel plug, effectively blocking passage of odorant molecules. In addition, local anesthetic in the formulation caused sufficient blocking of olfactory nerve impulses, and antihistamine in the formulation sufficiently dried mucosa, so that the subject's sensation of food-derived odorants was greatly diminished, leading to a decreased desire to consume food.

A formulation such as is disclosed herein is administered to a human subject to suppress said subject's appetite. The subject self-administers ad libitum a prototype formulation such as is described above. The subject sprays the formulation into each of her nasal cavities. The formulation adheres to the subject's nasal mucosa in the superior portion of the subject's nasal cavity. The formulation gels in situ in the superior portion of the nasal cavity and forms a gel plug, effectively blocking passage of odorant molecules. In addition, local anesthetic in the formulation causes sufficient blocking of olfactory nerve impulses, and antihistamine in the formulation sufficiently dries mucosa, so that the subject's sensation of food-derived odorants is greatly diminished, leading to a decreased desire to consume food.

A subject such as is described in either of the two paragraphs immediately above routinely self-administers ad libitum over the course of months a formulation such as is described above. The subject experiences diminished appetite and generally consumes lesser quantities of food than before he or she began the self-administration. The subject continues activity at a level approximately the same as prior to the self-administration. Due to the consumption of lesser quantities of food, the subject experiences a weight loss over the course of the period of self-administration of the formulation.

Subsequent to manufacture, a formulation such as is disclosed herein is placed in a suitable container-closure system for intranasal administration such as is known in the art. Processes for filling such a container-closure system with a composition having a viscosity such as that of a formulation disclosed herein are known in the art.

Many modifications and other embodiments of a composition and/or process such as is described in various embodiments herein will come to mind to one skilled in the art to which this disclosed composition and/process pertains having the benefit of the teachings presented in the foregoing description. Therefore, it is to be understood that a composition and/process such as is described in various embodiments herein is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A process for inducing anosmia in a human subject, the process comprising administering to the subject a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

2. The process of claim 1, wherein the histamine antagonist is azelastine or a pharmaceutically acceptable salt of azelastine or olopatadine or a pharmaceutically acceptable salt of olopatadine.

3. The process of claim 2, wherein the histamine antagonist is azelastine or a pharmaceutically acceptable salt of azelastine.

4. The process of claim 3, wherein the histamine antagonist is a pharmaceutically acceptable salt of azelastine.

5. The process of claim 4, wherein the pharmaceutically acceptable salt of azelastine is azelastine hydrochloride.

6. The process of claim 1, wherein the local anesthetic is lidocaine or a pharmaceutically acceptable salt of lidocaine.

7. The process of claim 6, wherein the pharmaceutically acceptable salt of lidocaine is lidocaine hydrochloride.

8. The process of claim 1, wherein the temperature greater than ambient temperature is a temperature obtained in a nasal cavity of the subject.

9. The process of claim 1, wherein the temperature greater than ambient temperature is a temperature from 34 degrees Celsius to 40 degrees Celsius.

10. The process of claim 1, wherein the temperature greater than ambient temperature is 34 degrees Celsius.

11. The process of claim 1, wherein the temperature greater than ambient temperature is 34.5 degrees Celsius.

12. The process of claim 1, wherein the temperature greater than ambient temperature is 37 degrees Celsius.

13. The process of claim 1, wherein the pharmaceutical composition is contained in an aerosolizing pump suitable for intranasal administration of a pharmaceutical liquid.

14. A process for inducing anosmia in a human subject, the process comprising administering to the subject a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

15. The process of claim 14, wherein the histamine antagonist is azelastine or a pharmaceutically acceptable salt of azelastine or olopatadine or a pharmaceutically acceptable salt of olopatadine.

16. The process of claim 15, wherein the histamine antagonist is azelastine or a pharmaceutically acceptable salt of azelastine.

17. The process of claim 16, wherein the histamine antagonist is a pharmaceutically acceptable salt of azelastine.

18. The process of claim 17, wherein the pharmaceutically acceptable salt of azelastine is azelastine hydrochloride.

19. The process of claim 14, wherein the local anesthetic is lidocaine or a pharmaceutically acceptable salt of lidocaine.

20. The process of claim 19, wherein the pharmaceutically acceptable salt of lidocaine is lidocaine hydrochloride.

21. The process of claim 14, wherein the temperature greater than ambient temperature is a temperature obtained in a nasal cavity of the subject.

22. The process of claim 14, wherein the temperature greater than ambient temperature is a temperature from 34 degrees Celsius to 40 degrees Celsius.

23. The process of claim 14, wherein the temperature greater than ambient temperature is 34 degrees Celsius.

24. The process of claim 14, wherein the temperature greater than ambient temperature is 34.5 degrees Celsius.

25. The process of claim 14, wherein the temperature greater than ambient temperature is 37 degrees Celsius.

26. The process of claim 14, wherein the pharmaceutical composition is contained in an aerosolizing pump suitable for intranasal administration of a pharmaceutical liquid.

27. A process for treating obesity in a human subject, the process comprising administering to the subject a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

28. A process for treating obesity in a human subject, the process comprising administering to the subject a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.

29. A process for suppressing appetite in a human subject, the process comprising administering to the subject a pharmaceutical composition for intranasal administration, the composition capable of inducing temporary anosmia to suppress appetite temporarily in a subject, the composition comprising at least one member of the set consisting of a histamine antagonist and a local anesthetic, the composition further comprising one or more pharmaceutically acceptable excipients, wherein the composition is sprayable at ambient temperature, and wherein the composition is capable of forming a gel at a temperature greater than ambient temperature.

30. A process for suppressing appetite in a human subject, the process comprising administering to the subject a pharmaceutical composition for intranasal administration, the composition capable of inducing temporary anosmia to suppress appetite temporarily in a subject, the composition comprising pectin, methylcellulose, microcrystalline cellulose, sodium carboxymethylcellulose, and glycerol ester of hydrogenated rosin, and optionally at least one member of the set consisting of a histamine antagonist and a local anesthetic, the composition further optionally comprising one or more additional pharmaceutically acceptable excipients, wherein the composition is sprayable at ambient temperature, and wherein the composition is capable of forming a gel at a temperature greater than ambient temperature.
